(19) United States
(12) Patent Application Publication
(10) Pub. No.: US 2026/0000277 A1
(43) Pub. Date: Jan. 1, 2026

(54) MEDICAL DEVICES, SYSTEMS, AND RELATED METHODS FOR TISSUE MANIPULATION

(71) Applicant: Boston Scientific Scimed, Inc., Maple Grove, MN (US)

(72) Inventors: Colby HARRIS, Norfolk, MA (US); Ryan V. WALES, Shrewsbury, MA (US)

(73) Assignee: Boston Scientific Scimed, Inc., Maple Grove, MN (US)

(21) Appl. No.: 19/252,544

(22) Filed: Jun. 27, 2025

Related U.S. Application Data
(60) Provisional application No. 63/666,277, filed on Jul. 1, 2024.

(51) Int. Cl. A61B 1/00

(52) U.S. Cl. CPC A61B 1/00137; A61B 1/00094

(57) ABSTRACT

A medical device and a medical system include a cap body with a wall forming a lumen and a working channel insert positioned within the lumen. The working channel insert includes a main body and a cavity, and a flange extending radially inward from an inner surface of the wall of the cap body. The flange is positioned at a distalmost portion of the working channel insert. An outer surface of the main body of the working channel insert and the inner surface of the wall of the cap body form a longitudinally extending gap.

### CROSS-REFERENCE TO RELATED APPLICATIONS

This application claims the benefit of priority to U.S. Provisional Application No. 63/666,277, filed on Jul. 1, 2024, which is incorporated by reference herein in its entirety.

### TECHNICAL FIELD

Aspects of this disclosure generally relate to medical devices, systems, and related methods used for facilitating tissue manipulation. Some aspects relate to medical devices, systems, and methods for facilitating the use of various medical tools during a medical procedure associated with manipulating a treatment site.

### BACKGROUND

Medical devices, such as endoscopes or other suitable insertion devices are employed for various medical procedures. For example, an elongated tool or endoscope may be used during Endoscopic Mucosal Resection (EMR) and Endoscopic Sub-mucosal Dissection (ESD) to resect a lesion from a target site (e.g., a mucosa or surface layer) attached to an underlying portion of tissue (e.g., a submucosa or underlying layer of dense irregular connective tissue). During these medical procedures, a cutting tool, such as a blade, snare, wire loop, or similar tool, may be extended from a working channel of the endoscope to perform tissue resection. Various devices may also be used during these medical procedures to manipulate the tissue. For example, devices are used to improve visibility at the target site, generate tissue traction, and/or provide suction during endoscopic mucosal dissection/resection.

However, users may spend time removing devices (e.g., a cutting tool) from the working channel to introduce other devices necessary to conduct other steps during the medical procedure. For example, a user may remove a cutting tool from the working channel to provide suction if a tissue begins to bleed, to remove fluid or particulate, etc. Afterward, users may pass devices (e.g., clips, coagulation graspers) distally through the working channel to manage bleeding or otherwise treat the target site. These additional steps may increase the procedure's costs, duration, and/or risks. Therefore, there is a need for a device, system, or method that may assist the user in minimizing device exchanges and/or enable the user to use multiple devices during a medical procedure.

The devices, systems, and methods of this disclosure may help to rectify one or more of the issues described above or address other aspects of the art.

### SUMMARY

In one example, a medical device may comprise a cap body with a wall forming a lumen and a working channel insert positioned within the lumen. The working channel insert may include a main body and a cavity, and a flange may extend radially inward from an inner surface of the wall of the cap body, positioned at a distalmost portion of the working channel insert. An outer surface of the main body of the working channel insert and the inner surface of the wall of the cap body may form a longitudinally extending gap.

Any examples described herein may have any of these features alone or in combination. The inner surface of the wall of the cap body may include a drainage hole, and the working channel insert may be proximal to the drainage hole. The main body of the working channel insert may extend proximally from the flange within the lumen of the cap body. The main body of the working channel insert may include a proximal edge, and the main body of the working channel insert may extend proximally past a proximal edge of the cap body such that the proximal edge of the working channel insert is positioned proximally to the proximal edge of the cap body. The longitudinally extending gap may extend proximally along a length of the main body of the working channel insert. The working channel insert may have a higher durometer than the wall of the cap body. The flange may extend radially outward from a proximal edge of the main body of the working channel insert, and wherein the flange forms a lip that surrounds a portion of the working channel insert. The main body of the working channel insert may be cantilevered and supported by the flange.

The working channel insert may include a proximal outer diameter and a distal outer diameter, and the proximal outer diameter may be smaller than the distal outer diameter. The working channel insert may include a proximal inner diameter and a distal inner diameter, and the proximal inner diameter may be larger than the distal inner diameter. The cap body may include a notch in a distal end of the cap body, and the notch may be configured to receive an accessory device tube. Furthermore, the medical device may include an opening in a portion of the cap body that is configured to receive a suction tube. The inner surface of the wall of the cap body may include a drainage hole and the notch and the drainage hole may be circumferentially approximately opposite to one another. The opening for the suction tube may be circumferentially spaced apart from the drainage hole. A portion of the drainage hole and a portion of the opening of the suction tube may overlap relative to a longitudinal axis of the cap body.

In another example, a medical system may comprise a shaft having a proximal end and a distal end, including a working channel. A cap may be configured to be coupled to the distal end of the shaft. The cap may include a cap body with a wall forming a lumen. The cap may include a notch in a distal end of the cap body configured to receive an accessory device tube. The cap may include an opening in a portion of the cap body spaced away proximally of the distal end of the cap body and configured to receive a suction tube. The cap may include a working channel insert positioned within the lumen of the cap, and the working channel insert may include a main body and a cavity. The cap may include a flange that may extend radially inward from an inner surface of the wall of the cap body and may be coupled to a distal portion of the working channel insert. The working channel insert may be positioned within a distal portion of the working channel to couple the cap to the shaft, and a distal face of the shaft may abut against a proximal face of the flange.

Any of the examples disclosed herein may include any of the following features in any combination. The suction tube may be coupled to the opening, and a distal end of the suction tube may be entirely within the lumen of the cap body. The system may include an accessory device tube coupled to the notch, and a distal end of the accessory device tube may extend distal of the cap body. The accessory device tube and the suction tube may be circumferentially spaced from each other between 150 degrees to 180 degrees along the wall of the cap body. The inner surface of the wall of the cap body may include a drainage hole, and the notch and the drainage hole may be circumferentially approximately opposite to one another.

In another example, a medical system may comprise a cap. The cap may include a cap body with a wall forming a lumen and a notch in a distal end of the cap body. The cap body may include an opening in a portion of the cap body spaced away proximally of the distal end of the cap body and may be configured to receive a suction tube. A working channel insert may be positioned within the lumen of the cap, and the working channel insert may include a main body, a cavity, and a flange. The flange may extend radially inward from an inner surface of the wall of the cap body and may be coupled to a distal portion of the working channel insert. The system may include an accessory device tube, and a distal portion of the accessory device tube may be received within the notch in the distal end of the cap body. The system may include a suction tube, and a distal portion of the suction tube may be received within the opening.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 is a side view of a medical device, according to an embodiment.

FIG. 2 is a perspective and partially transparent view of a cap of a medical device, according to an embodiment.

FIG. 3 is a perspective and partially transparent view of another cap of a medical device, according to aspects of this disclosure,

FIG. 4A is a partial cross-sectional side view of the cap of FIG. 3, according to aspects of this disclosure.

FIG. 4B is a partial cross-sectional side view of the cap of FIG. 3 coupled to a distal end of a medical device, according to aspects of this disclosure.

FIG. 5A is a cross-sectional view of the cap of FIG. 4A, according to aspects of this disclosure.

FIG. 5B is a cross-sectional view of the cap of FIG. 4B coupled to the distal end of the medical device, according to aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately,” are used to indicate a possible variation of ±10% in a stated value or characteristic. For ease of description, portions/regions/ends of the device and/or its components are referred to as proximal and distal ends/regions.

It should be noted that the term “proximal” refers to ends/regions closer to a user of the device, and the term “distal” is used herein to refer to ends/regions further away from the user. Similarly, saying that a component extends “distally” indicates that a component extends in a distal direction (away from a user), and saying that a component extends “proximally” indicates that a component extends in a proximal direction (toward a user).

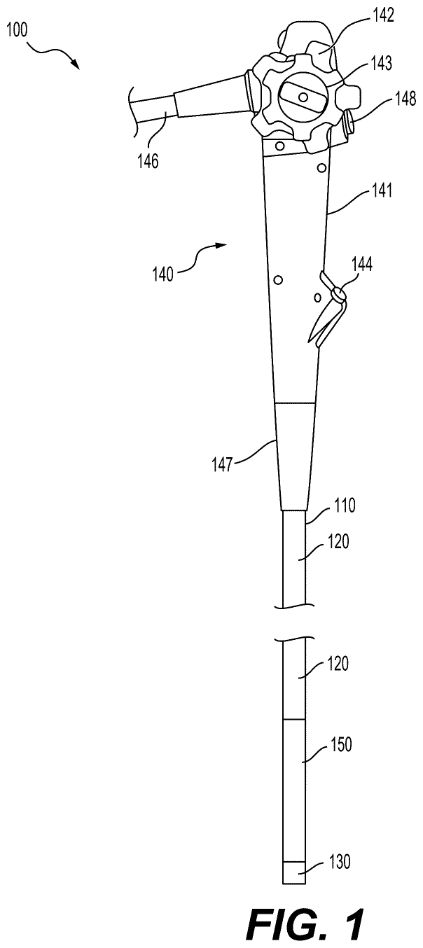

Referring to FIG. 1, a medical device 100, according to an embodiment, is shown. Medical device 100 may be an exemplary endoscope or insertion device. For example, medical device 100 may be user to deliver one or more devices for resecting polyps, lesions, tissue samples, or other undesired tissue from the interior bodily walls of a patient. However, any other medical device (e.g., bronchoscope, colonoscope, ureteroscope, duodenoscope, gastroscope, colonoscope, laparoscope, aspiration scope, etc.), catheter, sheath, or the like may be used in combination with aspects of this disclosure.

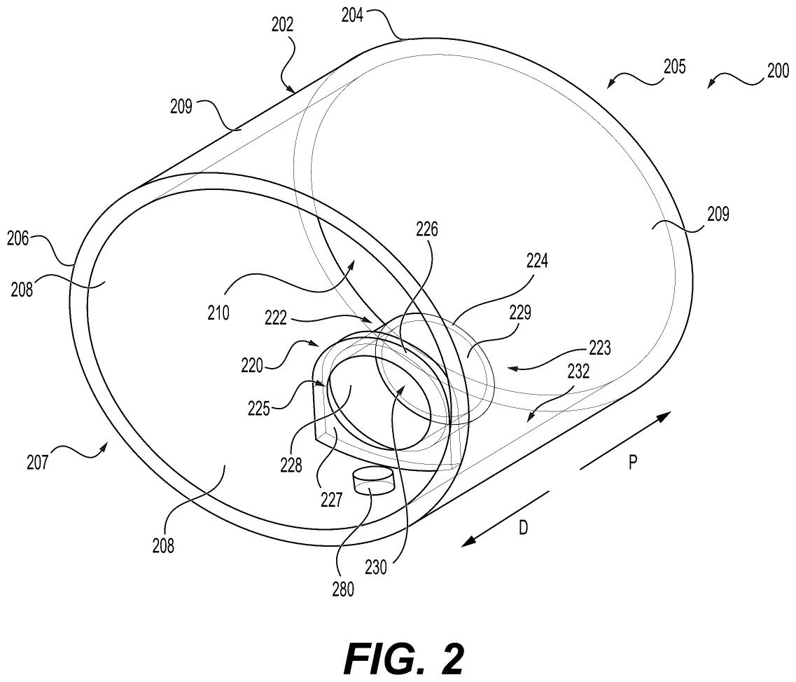

Medical device 100 includes a shaft 120 having a proximal end 110, a distal end 130, and at least one lumen extending between proximal end 110 and distal end 130. As described below, proximal end 110 of shaft 120 may be coupled to a handle 140, and distal end 130 of shaft 120 may be coupled to a cap 200 (FIG. 2). Shaft 120 may be any flexible or rigid member adapted to be advanced into a patient. In addition, shaft 120 may be flexible in some portions and rigid in others. For example, shaft 120 may include an articulation section 150 and a distal end 130 that may be flexible or steerable, helping shaft 120 to traverse circuitous cavities or lumens. In contrast, the rest of shaft 120 may be rigid to help urge shaft 120 distally into a body lumen.

Handle 140 may include a port 144 that may be configured to introduce and/or remove tools, fluids, or other materials to and from the shaft 120 for delivery to a target site and may be fluidically connected with shaft 120. In some aspects, port 144 may be positioned on a proximal portion 141 of handle 140. In other aspects, port 144 may be positioned on an intermediate and/or distal portion 147 of handle 140. In any of these aspects, port 144 may be fluidly connected to the lumen of shaft 120. In addition, an umbilicus 146 may be coupled to handle 140, for example, for providing fluid, suction, power, etc. to one or more aspects of medical device 100. Handle 140 may include one or more actuators, for example, a first actuator 142 and a second actuator 143.

A plurality of actuating elements (not shown), such as cables or wires, may extend distally from a proximal end of medical device 100 (such as handle 140) to articulation section 150 and/or distal end 130. For example, articulation/actuation wires may be indirectly coupled to an actuator, for example, first actuator 142 and/or second actuator 143, which may control articulation section 150 and/or distal end 130 in multiple directions (e.g., four-way steering). First actuator 142 and/or second actuator 143 may be, for example, rotatable knobs that rotate relative to handle 140 to push/pull actuating elements. Actuation of first actuator 142 and/or a second actuator 143 may control articulation section 150, distal end 130, and/or elements attached to distal end 130.

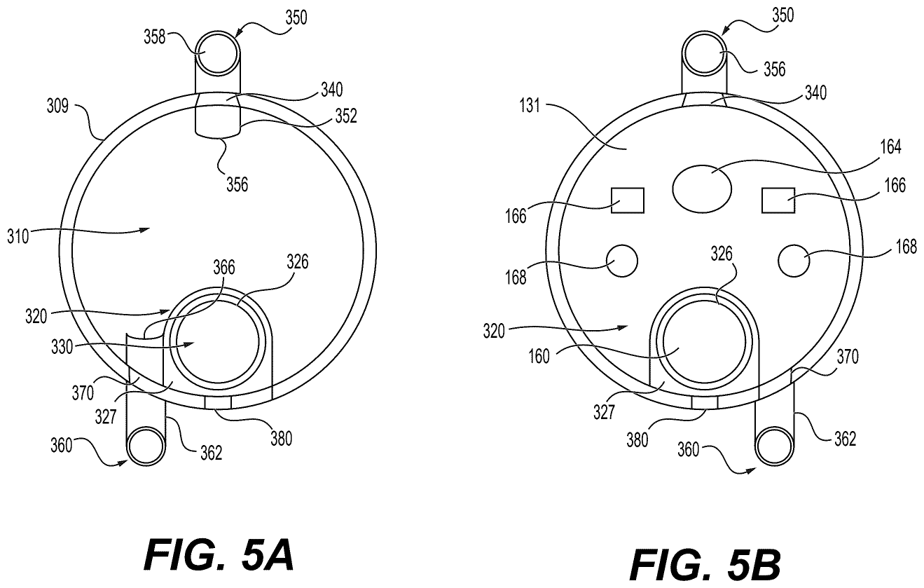

As mentioned, shaft 120 may include a lumen including one or more channels. Channels may have any cross-sectional geometry, including, but not limited to, circular, square, triangular, or polygonal. Further, some channels may include permanently fixed medical devices, while other channels may help the insertion of exchangeable medical devices. As shown in a cross-sectional view of FIG. 5B looking proximally, an exemplary distal face 131 of medical device 100 may include a plurality of openings of the one or more channels. For example, shaft 120 may include a working channel 160. A user may introduce (e.g., via port 144) one or more tools (e.g., cutting tools, such as a blade, knife, electrode, snare, wire loop, etc.) that extend out of working channel 160 at distal end 130. Shaft 120 may also include one or more fluid channel(s) 168. Fluid channels 168 may receive fluid, initially, by handle 140 being coupled to a fluid source (not shown) via a “fluid port” (not shown) or via umbilicus 146 to allow for a proximal fluid connection that delivers a pressured flow to fluid channel(s) 168. The fluid port may be positioned on a proximal portion of handle 140, for example, a proximal end of handle 140.

Furthermore, the medical device 100 may incorporate an imaging element 164 or an imager (e.g., a camera) for direct visualization of a patient's internal anatomy. Imaging element 164 may be positioned at distal face 131. Distal face 131 may also include one or more illuminators 166. Electrical cables (not shown) may extend from the proximal end of medical device 100 (such as via umbilicus 146) to distal end 130, providing electrical controls to imaging element 164, illuminator(s) 166, and/or other electrical devices in or on the distal face 131 of shaft 120. Imaging signals may be transmitted proximally from imaging element 164 by these cables to be processed and/or displayed on a display. In certain instances, one or more viewing element(s) 164 can be controlled via handle 140 by a user of medical device 100, such as via an imaging element 148 of handle 140.

FIG. 2 shows an embodiment of a cap 200. Cap 200 may be generally tubular and sized to fit or otherwise be coupled to distal end 130 of shaft 120. Cap 200 may include a working channel insert 220, a wall 202, and a lumen 210. As discussed below, at least a portion of lumen 210 may be configured to receive distal end 130 of shaft 120.

Wall 202 may be tubular and generally cylindrical, for example, defining the shape of cap 200. Wall 202 may include a circular cross-section, or a cross-section adapted to body lumens and/or a shape of shaft 120. Wall 202 may include an inner surface 208 and an outer surface 209. Outer surface 209 may be uniformly cylindrical along its length, and an inner surface 208 may define lumen 210, as described below. In other words, wall 202 may be a body of cap 200. The size of cap 200 may vary based on the size of the body lumens in which cap 200 and shaft 120 operate. For example, the size of cap 200 may be defined by a thickness of wall 202 (i.e., thickness between the outer surface 209 and inner surface 208) and a diameter or shape of lumen 210 defined by inner surface 208.

Furthermore, wall 202 may include a drainage hole 280, for example, extending from inner surface 208, through wall 202, to outer surface 209. In an embodiment, cap 200 may include a plurality of drainage hole(s) 280. In yet another embodiment, cap 200 may not include drainage hole 280 (i.e., for use in a procedure using suction only and/or when drainage from cap 200 is not needed). Drainage hole 280 may be positioned distal to and at least partially aligned (e.g., circumferentially aligned) with working channel insert 220.

Wall 202 may include a distal edge 206 and a proximal edge 204. Distal edge 206 and a proximal edge 204 may be beveled or smoothened, such that cap 200 is atraumatic to the surrounding tissue when inserted into a body lumen along with distal end 130 of shaft 120, as described below. Distal edge 206 may define a distal opening 207, and a proximal edge 204 may define a proximal opening 205. Proximal opening 205 and distal opening 207 may be circumferential, corresponding to the tubular shape of wall 202.

Lumen 210 of cap 200 may be a substantially empty or open space extending longitudinally from distal edge 206 (i.e., including distal opening 207) to proximal edge 204 (i.e., including proximal opening 205). As discussed, lumen 210 may be defined by inner surface 208 of wall 202. The shape of inner surface 208 may form the shape of lumen 210. For example, inner surface 208 may be tubular, resulting in lumen 210 also being tubular. The size of the inner surface 208 may also include a diameter/width sufficient for lumen 210 to accommodate a corresponding diameter/width of distal end 130 of shaft 120. In an embodiment, lumen 210 may have a snug fit (e.g., a friction fit, a snap fit, etc.) when radially surrounding at least a portion of distal end 130 of shaft 120, as described below.

Cap 200 may also include working channel insert 220. Working channel insert 220 may be proximal to drainage hole 280 on inner surface 208 of wall 202. Working channel insert 220 may include a distal flange 226, and a main body 222, and a cavity 230. Main body 222 may include a proximal edge 224, and a cavity 230. Main body 222 of working channel insert 220 may include an inner surface 228 and an outer surface 229. As described below, outer surface 229 of main body 222 may not touch or contact inner surface 208 of cap 200. Main body 222 of working channel insert 220 may be tubular and generally cylindrical. For example, outer surface 209 of main body 222 may be uniformly cylindrical along its length.

Main body 222 may include a circular cross-section or a cross-section adapted to be at least partially received within working channel 160 of shaft 120. Furthermore, proximal edge 224 may define a proximal opening 223 of working channel insert 220. Distal flange 226 may be positioned at the distalmost end of working channel insert 220, distal of main body 222, and may define a distal opening 225 of working channel insert 220. Cavity 230 of working channel insert 220 may be a substantially empty or open space extending longitudinally from proximal edge 224 (i.e., including proximal opening 223) to distal flange 226 (i.e., including distal opening 225). The shape of inner surface 228 may form the shape of cavity 230. For example, inner surface 228 may be tubular, resulting in cavity 230 being tubular.

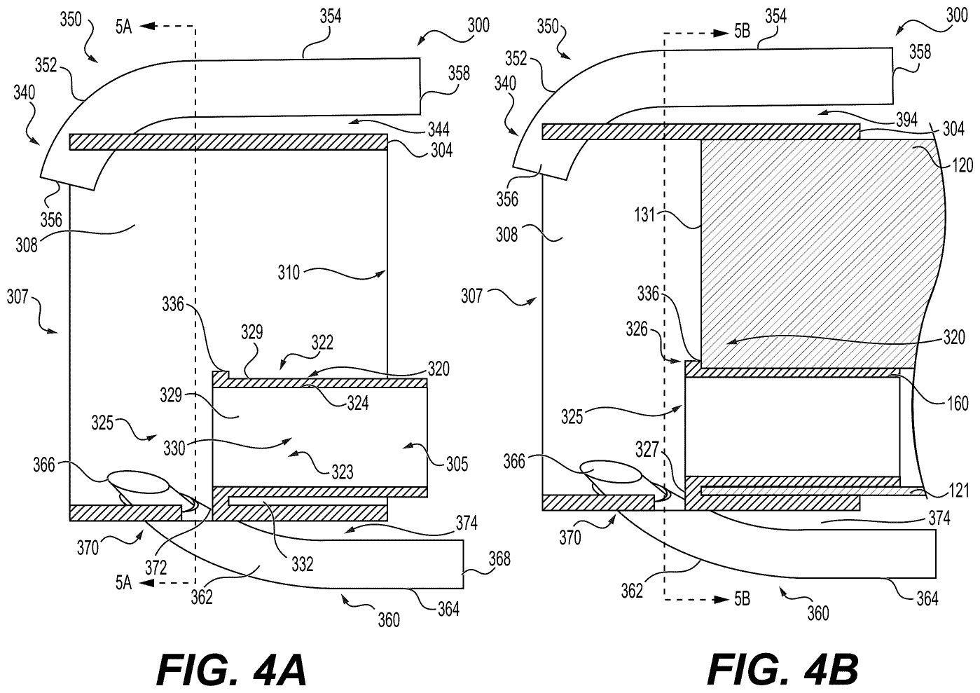

Distal flange 226 may be an angular, semi-annular portion of working channel insert 220. Distal flange 226 may be larger relative to the other components of working channel insert 220 and include a lip 227 or a stop surface that surrounds distal flange 226. In other words, distal flange 226 (i.e., including lip 227) may be the largest portion of working channel insert 220. The components proximal to distal flange 226, such as main body and proximal edge 224, may be smaller in comparison to distal flange 226. Further features, of cap 200, such as a gap 232 may be better seen in reference to a cap 300 in FIGS. 4A-4B and FIGS. 5A-5B. Although, FIGS. 4A-4B show an embodiment of a cap 300, cap 300 is similar to cap 200 except for the differences described below (i.e., additional structures). Accordingly, reference numerals adding “100” to the reference numbers used to describe cap 200 are used to identify substantially similar components of cap 300.

Distal flange 226 (e.g., similar to a distal flange 326) may extend radially inward, such that gap 232 (e.g., similar to a gap 332) is formed between working channel insert 220 and inner surface 208 of wall 202. Distal flange 226 may define the distalmost point of gap 232. Gap 232 extends proximally along the length of main body 222 to proximal edge 224, which is the proximal-most point of main body 222 or to proximal edge 204 of wall 202, which is the proximal-most point of wall 202. As shown in FIGS. 4A-4B but described in reference to cap 200, main body 222 of working channel insert 220 (e.g., a main body 322 of a working channel insert 320) may be cantilevered, such that distal flange 226 (e.g., a distal flange 226) and lip 227 are the only part of working channel insert 220 to contact or connect to inner surface 208 of wall 202. In other words, main body 222 is supported at only one end (i.e., the end including distal flange 226 and lip 227), and may extend longitudinally, in a proximal direction, without additional support. Therefore, main body 222 may extend longitudinally from distal flange 226, ensuring outer surface 229 of main body 222 does not touch wall 202 (i.e., inner surface 208 of wall 202). In an embodiment, main body 222 extends up to proximal edge 204 of cap 200 (i.e., remain in lumen 210). In another embodiment, main body 222 of working channel insert 220 may extend proximally past or beyond proximal edge 204, extending past cap 200, and helping working channel insert 220 be introduced into a longer length of working channel 160 of shaft 120.

Cap 200 or cap 300 may be manufactured using any suitable method, such as a molding process (e.g., a soft elastomer mold), and may be made from various materials, including elastics, rubbers, plastics, or any suitable flexible polymer. Additionally, cap 200 including working channel insert 220 and cap 300 including working channel insert 320 may be manufactured with varying durometers or rigidities according to the requirements of different medical procedures. For example, working channel insert 220 or working channel insert 320 may have a higher durometer than its respective cap 200 or cap 300. A working channel insert 220 or working channel insert 320 with a higher rigidity may help maintain its position within working channel 160 of distal end 130, for example, helping to maintain a connection between cap 200, 300 and distal end 130 during delivery to the target site and/or while medical tools pass through working channel 160 and working channel insert 220, 320. In some embodiments, one or more portions of cap 200 or cap 300 may be expandable (i.e., have a lower durometer than working channel insert 220 or working channel insert 320), allowing users to attach cap 200 or cap 300 to shaft 120 of medical device 100 with varying diameters or designs. For instance, wall 202 of cap 200 (or wall 302 of cap 300) may be made of a flexible material, which may expand radially to fit around shaft 120 even with shaft 120 having a diameter greater than the diameter of cap 200 or cap 300. Cap 200 or cap 300 may also return to a relaxed position to fit around shaft 120 having a diameter comparable with the diameter of cap 200 or cap 300. Conversely, cap 200 or cap 300 may have a higher durometer, making it less flexible than working channel insert 220 or working channel insert 320.

The function of the embodiment of cap 200 of FIG. 2 will be described in conjunction with the embodiment shown in FIGS. 4A-4B. In other words, cap 200 (FIG. 2) and cap 300 (FIGS. 4A-4B) may be introduced over distal end 130 of shaft 120, used similarly, and include similar advantages where applicable (i.e., when including the same structures). First, a user may introduce cap 200 or cap 300 over distal end 130 of shaft 120. Distal edge 206 of cap 200 (or distal edge 306 of cap 300) optionally include threading, projections, grooves, or any suitable means to attach to shaft 120. Attachment mechanisms (not shown) in the disclosure may include, but are not limited to, friction-fit, snap-fit, or compression-fit arrangements.

Distal end 130 of shaft 120 may be coupled to cap 200, 300, such that working channel insert 220 or working channel insert 320 (if using cap 300) aligns with the opening of working channel 160 of shaft 120. As illustrated in FIG. 4B, as shaft 120 is introduced, distal face 131 of distal end 130 may abut against a proximal surface of distal flange 226 (or a proximal surface 336 of distal flange 326). Once distal face 131 of shaft 120 abuts distal flange 226 (or distal flange 326), shaft 120 cannot be advanced further distally past working channel insert 220 (or working channel insert 320).

As shown in FIG. 4B, distal face 131 may abut against lip 327 of a distal flange 326 (similar to lip 227 of distal flange 226). Once distal face 131 abuts distal flange 226 or distal flange 326, working channel insert 220 or working channel insert 320 may be introduced into working channel 160. When introduced, main body 222 of working channel insert 220 (or main body 322 of working channel insert 320) may align with the inner diameter (ID) of working channel 160 at the distal end 130. As shown in FIG. 4B, outer surface 329 of main body 322 (or outer surface 229 of main body 222) may align with the inner diameter of working channel 160 when distal face 131 abuts distal flange 226 (i.e., also lip 227).

Medical tools extended through working channel 160 may be extended through cavity 230 of working channel insert 220 (or cavity 330 of working channel insert 320). The remainder of distal face 131, for example, imaging element 164 and illuminators 166, may be positioned within lumen 210 or lumen 310 (i.e., outside of working channel insert 220 or working channel insert 320). Additionally, cap 200, 300 may not interfere with the operation and/or visualization of imaging element 164 and illuminators 166.

Cap 200 or cap 300 may help users identify the angular positioning and longitudinal distance of the distal face 131 relative to distal edge 206 or the distal-most point of lumen 210 (or distal edge 306 or the distal-most point of lumen 310 if using cap 300). The distance may be measured from distal flange 226 or distal flange 326 (i.e., acting as a reference point), which helps to prevent distal face 131 from moving proximally and abutting distal edge 206 of cap 200 (or distal edge 306 of cap 300). Therefore, cap 200 or cap 300 may include a fixed distance between distal face 131 and the distal-most point of cap 200 or cap 300 (i.e., a portion of cap contacting tissue walls of a body lumen). As a result, flange 226 and working channel insert 220 and flange 326 and working channel insert 320 may define the longitudinal orientation (e.g., relative to a longitudinal axis 399) of cap 300 (or cap 200). For example, in these aspects, cap 200 may help to maintain a distance or spacing of various components (e.g., fluid channel(s) 168 in FIG. 5B) of distal face 131 from distal edge 206 of cap 200 (or distal edge 306 of cap 300). The distance or spacing of various components may help to ensure that the various components of distal face 131 (e.g., imaging element 164, illuminators 16, etc.) do not abut or otherwise interfere with tissue or other portions of the target site, which could obstruct the view of a user.

Main body 222 of working channel insert 220 may include both a proximal outer diameter and a distal outer diameter, as defined by outer surface 229. Similarly, main body 222 may include a proximal inner diameter and a distal inner diameter, defined by inner surface 228. The dimensions of the outer diameter may be helpful, for example, to the insertion of working channel 160 of shaft 120 into the working channel insert 220. Conversely, the dimensions of the inner diameter may be relevant when accommodating devices within working channel 160 and preventing unintentional contact with a surface (i.e., distal flange 226). For example, an outer diameter of main body 222 may remain constant along the length of main body 222. However, a thickness of main body 222 of working channel insert 220 may decrease from the distal end to the proximal end. In this aspect, an inner diameter of main body 222 may increase from a distal end to a proximal end. Correspondingly, cavity 230 of main body 222 may enlarge from a distal end to a proximal end as main body 222 tapers in a proximal direction.

In some embodiments, working channel insert 220 and working channel insert 320 may taper radially inward, for example, moving in a proximal direction or in a distal direction relative to the position of working channel insert 220. As an example (e.g., also applicable to working channel insert 320), inner surface 228 of main body 222 may taper radially inward moving in a proximal direction. In the embodiment, the proximal outer diameter may be smaller than the distal outer diameter, and the proximal inner diameter may be smaller than the distal inner diameter. In some embodiments, the proximal outer diameter and the distal outer diameter may remain the same (e.g., consistent), while the proximal inner diameter may be smaller than the distal inner diameter. In other words, working channel insert 220 may be narrower at the proximal edge 224 than at the distal end of main body 222, and correspondingly the diameter of cavity 230 may be larger at the distal end than at the proximal end. In the embodiment, as the main body tapers proximally, the thickness of main body 222 of working channel insert 220 may increase from the proximal edge 224 towards distal flange 226.

Alternatively or additionally, and again using working channel insert 220 as an example (e.g., also applicable to working channel insert 320) inner surface 228 of main body 222 could expand radially outward, moving in a proximal direction. In the embodiment, the proximal outer diameter may be larger than the distal outer diameter and the proximal inner diameter may be larger than the distal inner diameter. In some embodiments, the proximal outer diameter and the distal outer diameter may remain the same, while the proximal inner diameter may still be larger than the distal inner diameter.

In other words, working channel insert 220 may be narrower at the distal end than at the proximal end, and correspondingly the diameter of cavity 230 would be larger at the proximal end than at the distal end. This tapering or narrowing may help working channel 160 be introduced into main body 222 (i.e., a proximal opening 223), but still fit (e.g., abut or fit flush) against flange 226. In the embodiment, as main body 222 tapers proximally, the thickness of main body 222 of working channel insert 220 may increase from distal flange 226 to proximal edge 224. Furthermore, in the embodiment, gap 232 may be larger toward the proximal end (e.g., proximal edge 204), which may facilitate insertion of distal end 130 of shaft 120 (i.e., a bottom portion 121 of shaft 120).

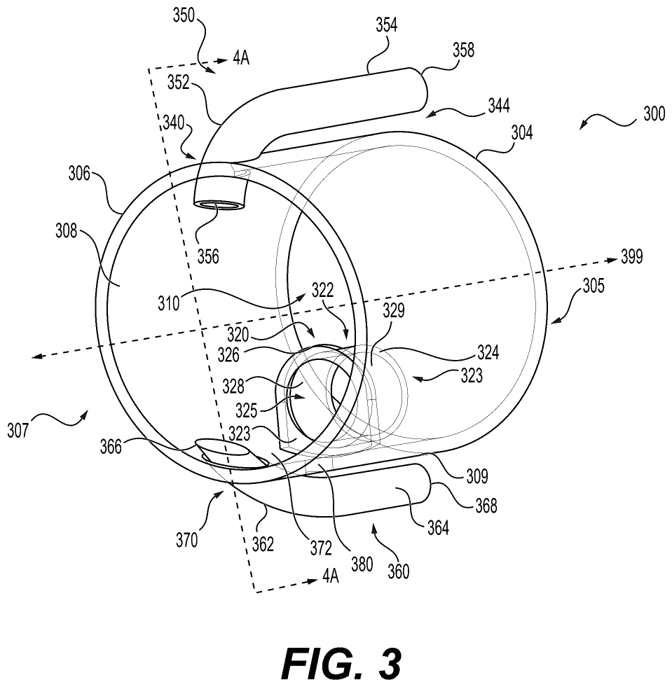

As previously discussed, cap 200 and cap 300 may share similar features unless otherwise specified. As shown in FIG. 3, cap 300 may also be generally tubular and sized to fit to distal end 130 of shaft 120. Cap 300 includes a working channel insert 320, a wall 302, and a lumen 310 configured to receive distal end 130 of shaft 120. However, cap 300 may incorporate additional features, such as an accessory device tube 350 and a suction tube 360. Cap 200 may be modified to include the features described in relation to cap 300.

Cap 300 may include at least one indentation or a notch 340. In some embodiments, multiple notches 340 may be positioned on cap 300, for example, circumferentially around portions of cap 300. Notch 340 may be a U-shaped or semi-annular slot, recess, or indentation positioned on a distal edge 306 of cap 300. The dimensions of notch 340 may be tailored to receive accessory device tube 350 and snugly accommodate or otherwise at least temporarily secure a portion of accessory device tube 350. Furthermore, notch 340 may be approximately circumferentially opposite to a drainage hole 380 (i.e., similar to drainage hole 280).

Accessory device tube 350 may be a tube (e.g., a cylindrical tube), including a curved segment 352 and a linear segment 354. A gap 344 may be formed between accessory device tube 350 and outer surface 309 of wall 302. The inner curvature of notch 340 may match the outer diameter of accessory device tube 350, ensuring a secure attachment to cap 300. Accessory device tube 350 may be removably or fixedly attached to notch 340 in any suitable way, which may include, but is not limited to, friction-fit, snap-fit, crimping, or compression-fit arrangements. The depth of notch 340 may be equal to or slightly less than the radius of accessory device tube 350, creating a stable seating area that helps to prevent movement. The edges of notch 340 may be smooth and/or rounded to facilitate insertion of accessory device tube 350.

Accessory device tube 350 may include a distal opening 356, and a proximal opening 358. In an embodiment, accessory device tube 350 may extend proximally to be adjacent to, for example, handle 140 of medical device 100. Proximal opening 358 may be on a proximalmost end of a linear segment 354, while distal opening 356 may be positioned at a distalmost end of curved segment 352 of accessory device tube 350. When accessory device tube 350 is installed on cap 300, a portion of curved segment 352 and distal opening 356 may extend at-least partially distal of distal opening 307 of cap 300. Proximal opening 358 may be used to introduce a variety of medical tools into accessory device tube 350, for example, coagulation graspers, clips, and/or any medical tool necessary for a procedure. Medical tools may be delivered through the lumen of accessory device tube 350 and extend past distal opening 356 (i.e., distal, of distal opening 356).

Cap 300 may also include an opening 370 within wall 302 of cap 300 and suction tube 360. Opening 370 may be an annular opening or recess extending from an inner surface 308 to an outer surface 309 of wall 302. The dimensions of opening 370 may be tailored to receive suction tube 360. Opening 370 may be formed and/or positioned to be proximal to working channel insert 320. For example, a space 372 may be formed between opening 370 (i.e., and as described below, a portion of suction tube 360) and working channel insert 320. Space 372 may provide a distance from working channel insert 320 and components extending from working channel 160 of shaft 120, as seen in FIG. 4B. Additionally, opening 370 may be circumferentially spaced away proximally from distal edge 306 and circumferentially spaced apart from drainage hole 380. Furthermore, a portion of drainage hole 380 and a portion of opening 370 may overlap relative to longitudinal axis 399 of cap 300.

Suction tube 360 may be a cylindrical tube configured to provide suction, including a curved segment 362 and a linear segment 364. In some aspects, suction tube 360 may be used to deliver irrigation fluid, air, or other substances from a proximal end to a distal end, rather than applying suction or negative pressure to draw substances from the distal end toward the proximal end. As shown in FIGS. 4A and 4B, a gap 374 may be formed between a portion of curved segment 362 of suction tube 360 and outer surface 309 of wall 302. Suction tube 360 may include a distal opening 366 and a proximal opening 368. Proximal opening 368 may be located at a proximalmost end of linear segment 364, while distal opening 366 may be situated at a distalmost end of curved segment 354 of suction tube 360. Proximal opening 368 of suction tube 360 may be connected (e.g., directly, or indirectly) to a medical suction device or vacuum system, providing the necessary suction force for various medical procedures. At least a drainage hole 380 (FIGS. 5A-5B) may be positioned proximate to working channel insert 320, similar to drainage hole 280. Suction may assist with removal of fluids or debris from a target site. Distal opening 366 of suction tube 360 may align with an opening 370 in wall 302 of cap 300 to accept the distal end of the suction tube 360 (i.e., including opening 366). When installed onto cap 300, distal opening 366 of suction tube 360 may be entirely within lumen 310 of cap 300.

In the embodiment, when suction tube 360 is installed on cap 300, a portion of curved segment 352, including distal opening 366, may be entirely within lumen 310 of cap 300. The positioning of suction tube 360 of cap 300 may thereby minimize the risk of inadvertently suctioning the tissue wall of a body lumen at the target site, thereby reducing potential trauma to nearby tissue. The positioning and/or angling of the distal opening 366 of suction tube 360 within lumen 310 of cap 300 may direct suction towards the sides of the lumen 310. The angle of distal opening 366 may enhance the efficiency of suction by, for example, targeting debris located along the walls of the lumen, reducing the likelihood of clogging by directing suction away from the center where larger tissue pieces may obstruct suction tube 360, and/or minimizing the risk of damaging the surrounding tissue by preventing direct suction against a tissue wall.

As shown, accessory device tube 350 and suction tube 360 may be diametrically or circumferentially opposite each other, approximately 150 to 180 degrees apart. However, various angular arrangements may be used. Cap 300 may include a plurality of notches 340 at different angular orientations, helping users select the most suitable arrangement for their specific needs. For instance, additional notches 340 could be positioned at 90-degree intervals, enabling configurations where accessory tube(s) 350 and suction tube(s) 360 are placed at 90, 180, or 270 degrees relative to each other. In other words, users may customize the setup of cap 300 according to the requirements of the medical procedure, such as positioning accessory tube(s) on various notches 340 to target specific areas within a body lumen with a medical tool.

As discussed, distal end 130 of shaft 120 may be introduced such that working channel insert 320 aligns with the opening of working channel 160 of shaft 120. As illustrated in FIG. 4B, as shaft 120 is introduced, distal face 131 of shaft 120 may abut against proximal surface 336 of distal flange 326. Once distal face 131 of shaft 120 abuts distal flange 226, shaft 120 cannot be advanced further proximally past working channel insert 320. By incorporating working channel insert 320 in cap 300, the angular positioning of the components of cap 300 may be established (i.e., similar to cap 200). For example, working channel insert 320 may provide a fixed reference point, such that a user may know the angular distance and orientation of fluid channel(s) 168, visualizing element 164, and working channel 116 relative to one another; this may help features of distal face 131 maintain a consistent alignment when abutting flange 326 of working channel insert 320. Furthermore, fixed angular distances (i.e., when cap 300 is fully assembled) between working channel insert 320 (i.e., the reference point) and accessory device tube(s) 350 may be established to assist with the positioning of notches 340. Therefore, a user may know the position of tools extending from accessory device tube(s) 350 relative to tools extending from working channel 116, helping to minimize the risk of tools disrupting each other during a procedure.

Furthermore, a kit may include multiple caps (i.e., cap 200 and/or cap 300) designed to accommodate a range of medical procedures, and medical devices with varying shaft(s) 120. Each cap in the kit may vary in one or more aspects to provide optimal compatibility and functionality. For example, caps may vary in diameter of their respective lumen (e.g., lumen 210 and/or lumen 310) to fit various shaft sizes. In addition, caps may vary in the diameter of their respective working channel insert (e.g., working channel insert 220 or working channel insert 320) to match the different working channel sizes of varying shafts available in the market. Furthermore, caps may include a range of notches (e.g., notch 340), ranging from one to multiple notches (e.g., notch 340) to accommodate accessory tube(s) 350 (e.g., several of accessory tube(s) 350). As described above, several caps in the kit may feature various angular orientations for the notches, helping to facilitate different placements of the accessory tubes to suit specific procedural needs. This variety may enable users to select the most suitable cap for their medical device and the medical procedure being performed.

In use, a medical device including a shaft (e.g., an endoscope) may be selected for a procedure, along with a suitable cap (e.g., cap 200 or cap 300, including the variations described above). A user may select the cap based on several features. For example, the diameter of the lumen (e.g., lumen 210, lumen 310) to ensure a snug fit over distal end 130 of shaft 120. The cap may also be selected based on features (e.g., diameter) of a working channel insert (e.g., working channel insert 220, working channel insert 320) that aligns with the working channel 160.

A user may introduce distal end 130 of shaft 120 into lumen 210 of cap 200 or lumen 310 of cap 300. The cavity 230 or cavity 330 of working channel insert working channel insert 220 or working channel insert 320 may be aligned with the opening of the working channel 160 of the shaft 120. As a user advances shaft 120, distal face 131 of shaft 120 may abut the proximal surface of distal flange 226 (or proximal surface 336 in cap 300).

After placing the cap over distal end 130 of shaft 120, as described above, the user may insert distal end 130 of the shaft into a body lumen and navigate the lumen to reach a treatment site. The user may insert medical tools as desired through a lumen of accessory tube(s) 350 (e.g., inserting a plurality of medical tools into additional accessory tube(s) 350). Additionally, the user may insert medical tools through a lumen of working channel 160, extending medical tools distally through working channel insert (e.g., working channel insert 220 or working channel insert 320) as needed. A user may also deliver fluid via fluid channel(s) 168 as needed to irrigate and/or provide traction to a target site. Alternatively, or additionally, the user may apply suction as desired through suction tube 360 to collect debris. Once the procedure is complete, shaft 120 may be retrieved from the body lumen, and the cap may be removed from the distal end 130 of shaft 120, and a user may dispose of the cap appropriately.

The integration of cap 200 and cap 300 described herein including features of, for example, working channel insert 220 and working channel insert 320, and accessory device tube 350 and suction tube 360, may resolve one or more challenges posed by other caps introduced onto a distal end of a medical device including a shaft. Furthermore, cap 200 and cap 300 may help to facilitate the use of additional medical tools used to manipulate tissue during a procedure.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a cap body with a wall forming a lumen;
a working channel insert positioned within the lumen of the cap body, wherein the working channel insert includes a main body and a cavity; and
a flange, extending radially inward from an inner surface of the wall of the cap body and positioned at a distalmost portion of the working channel insert;
wherein an outer surface of the main body of the working channel insert and the inner surface of the wall of the cap body form a longitudinally extending gap.

2. The medical device of claim 1, wherein the inner surface of the wall of the cap body includes a drainage hole, wherein the working channel insert is proximal to the drainage hole.

3. The medical device of claim 1, wherein the main body of the working channel insert extends proximally from the flange within the lumen of the cap body.

4. The medical device of claim 1, wherein the main body of the working channel insert includes a proximal edge, wherein the main body of the working channel insert extends proximally past a proximal edge of the cap body such that the proximal edge of the working channel insert is positioned proximally of the proximal edge of the cap body.

5. The medical device of claim 1, wherein the longitudinally extending gap extends proximally along a length of the main body of the working channel insert.

6. The medical device of claim 1, wherein the working channel insert has a higher durometer than the wall of the cap body.

7. The medical device of claim 1, wherein the flange extends radially outward from a proximal edge of the main body of the working channel insert, and wherein the flange forms a lip that surrounds a portion of the working channel insert.

8. The medical device of claim 7, wherein the main body of the working channel insert is cantilevered and supported by the flange.

9. The medical device of claim 1, wherein the working channel insert includes a proximal outer diameter and a distal outer diameter, wherein the proximal outer diameter is smaller than the distal outer diameter.

10. The medical device of claim 1, wherein the working channel insert includes a proximal inner diameter and a distal inner diameter, wherein the proximal inner diameter is larger than the distal inner diameter.

11. The medical device of claim 1, wherein the cap body includes a notch in a distal end of the cap body, wherein the notch is configured to receive an accessory device tube.

12. The medical device of claim 11, further comprising an opening in a portion of the cap body that is configured to receive a suction tube.

13. The medical device of claim 12, wherein the inner surface of the wall of the cap body includes a drainage hole, wherein the notch and the drainage hole are circumferentially approximately opposite to one another.

14. The medical device of claim 13, wherein the opening of the suction tube is circumferentially spaced apart from the drainage hole.

15. The medical device of claim 14, wherein a portion of the drainage hole and a portion of the opening of the suction tube overlap relative to a longitudinal axis of the cap body.

16. A medical system, comprising:
a medical device shaft having a proximal end and a distal end, including a working channel; and
a cap configured to be coupled to the distal end of the shaft, the cap including:
a cap body with a wall forming a lumen;
a notch in a distal end of the cap body configured to receive an accessory device tube;
an opening in a portion of the cap body spaced away proximally of the distal end of the cap body and configured to receive a suction tube;
a working channel insert positioned within the lumen of the cap, wherein the working channel insert includes a main body and a cavity; and
a flange, extending radially inward from an inner surface of the wall of the cap body and coupled to a distal portion of the working channel insert;
wherein the working channel insert is configured to be positioned within a distal portion of the working channel to couple the cap to the shaft, and wherein a distal face of the shaft abuts against a proximal face of the flange.

17. The medical system of claim 16, further comprising the suction tube and an accessory device tube, wherein, when the suction tube is coupled to the opening, a distal end of the suction tube is entirely within the lumen of the cap body, and wherein, when the accessory device tube is coupled to the notch, a distal end of the accessory device tube extends distal of the cap body.

18. The medical system of claim 16, wherein the accessory device tube and the suction tube are circumferentially spaced from each other by between 150 degrees to 180 degrees along the wall of the cap body.

19. The medical system of claim 16, wherein the inner surface of the wall of the cap body includes a drainage hole, wherein the notch and the drainage hole are circumferentially approximately opposite to one another.

20. A medical system, comprising:
a cap, the cap including:
a cap body with a wall forming a lumen;
a notch in a distal end of the cap body;
an opening in a portion of the cap body spaced away proximally of the distal end of the cap body and configured to receive a suction tube;
a working channel insert positioned within the lumen of the cap, wherein the working channel insert includes a main body and a cavity; and
a flange, extending radially inward from an inner surface of the wall of the cap body and coupled to a distal portion of the working channel insert;
an accessory device tube, wherein a distal portion of the accessory device tube is received within the notch in the distal end of the cap body; and
a suction tube, wherein a distal portion of the suction tube is received within the opening.
